(19) United States
(12) Patent Application Publication
(10) Pub. No.: US 2020/0080150 A1
(43) Pub. Date: Mar. 12, 2020

(54) NON-INVASIVE PREIMPLANTATION GENETIC SCREENING

(71) Applicant: THE REGENTS OF THE UNIVERSITY OF CALIFORNIA, OAKLAND, CA (US)

(72) Inventor: Mousa SHAMONKI, LOS ANGELES, CA (US)

(73) Assignee: THE REGENTS OF THE UNIVERSITY OF CALIFORNIA, OAKLAND, CA (US)

(21) Appl. No.: 15/746,783

(22) PCT Filed: Jul. 27, 2016
(86) PCT No.: PCT/US16/44297

§ 371 Date: Jan. 22, 2018

Related U.S. Application Data
(60) Provisional application No. 62/197,449, filed on Jul. 27, 2015.

(51) Int. Cl. C12Q 1/6883; G01N 33/68

(52) U.S. Cl. CPC C12Q 1/6883; C12Q 2600/156; G01N 33/689

(57) ABSTRACT

A method of producing a preparation of embryonic DNA that can be performed without risking damage to a developing embryo is described. A sample of cell-free media in which an embryo was grown in vitro from the 4-blastomeres stage to the blastocyst stage of development is obtained, wherein the sample comprises cell-free embryonic DNA (cfeDNA). cfeDNA is obtained from the sample and recovered. These steps can be carried out non-invasively, without performing a biopsy of the embryo. The method can be used for optimizing the likelihood of a live birth after implantation of human egg fertilized in vitro, and for reducing the risk of damage to a normal embryo that has been fertilized in vitro.

This application claims benefit of U.S. provisional patent application No. 62/197,449, filed Jul. 27, 2015, the entire contents of which are incorporated by reference into this application.

### TECHNICAL FIELD OF THE INVENTION

This invention relates to methods, kits and preparations that can be used to improve preimplantation genetic screening, including a method of producing a preparation of embryonic DNA that can be performed without risking damage to a developing embryo. The methods can be carried out non-invasively, without performing a biopsy of the embryo, providing methods for optimizing the likelihood of a live birth after implantation of human egg fertilized in vitro, and for reducing the risk of damage to a normal embryo that has been fertilized in vitro.

### BACKGROUND OF THE INVENTION

In vitro fertilization (IVF) involves stimulating a woman's eggs to grow and retrieving them from the body with a needle under anesthesia. The eggs are then fertilized in the laboratory and grown in media specifically designed for appropriate embryo development. As the embryos develop, they are commonly transported between different media. Eventually the highest quality embryo(s) are replaced into the uterine cavity while any remaining high quality embryos may be frozen for future use.

Embryos grown in this in vitro environment are often biopsied in order to test the chromosomes for a specific disorder, such as cystic fibrosis, or to test the embryo for genetic competency by whole chromosome analysis. These methods of preimplantation genetic diagnosis (PGD) or screening (PGS) involve making an opening through the outer shell of the egg (zona pellucida) about two days before the biopsy is performed. This biopsy is an invasive and expensive process.

There remains a need for improved methods to obtain preparations of embryonic DNA in order to screen and diagnose embryos for genetic and/or chromosomal competency that minimize the risks and expense associated with embryo biopsy.

### SUMMARY OF THE INVENTION

The invention provides a novel method of producing a preparation of embryonic DNA that can be performed without risking damage to a developing embryo. In one embodiment, the method comprises obtaining a sample of cell-free media in which an embryo was grown in vitro from the 4-10 blastomeres stage to the blastocyst stage of development, wherein the sample comprises cell-free embryonic DNA (cfeDNA). The method further comprises amplifying DNA obtained from the sample; and recovering the amplified cfeDNA. These steps can be carried out non-invasively, without performing a biopsy of the embryo. In one embodiment, the embryo is subjected to assisted hatching prior to obtaining the sample of media. In some embodiments, the sample of media is about 15-20 μl in volume. In some embodiments, the method further comprises analyzing copy number variations in the ploidy of the cfeDNA relative to a reference sample, or sequencing a target region of the DNA associated with a suspected genetic abnormality; and detecting the presence or absence of a genetic or chromosomal abnormality in the embryo. Representative examples of a chromosomal abnormality include, but are not limited to, a deletion or duplication of a chromosome. Representative examples of a genetic abnormality include, but are not limited to, a mutation associated with mortality or morbidity.

The analyzing optionally comprises labeling and denaturing the amplified DNA, and hybridizing the labeled, denatured DNA in a 1:1 ratio to a normal metaphase spread of chromosomes. In some embodiments, the analyzing comprises comparative genomic hybridization (CGH), fluorescence in situ hybridization (FISH), next generation gene sequencing, and/or giemsa banding. In one embodiment, the CGH is array CGH.

The invention further provides a method for optimizing the likelihood of a live birth after implantation of human egg fertilized in vitro comprising performing the method described above on a plurality of embryos developed after in vitro fertilization and selecting an embryo in which no genetic or chromosomal abnormality is detected. Also provided is a method for reducing the risk of damage to a normal embryo that has been fertilized in vitro comprising performing the method of the invention in lieu of trophectoderm biopsy on an embryo developed after in vitro fertilization.

### BRIEF DESCRIPTION OF THE DRAWINGS

FIG. 1. Illustration of preparation and collection of spent IVF media for non-invasive genetic screening. 1) On day-3 of embryonic development, each embryo was removed from G1 media, 2) underwent assisted hatching where an opening in the zona pellucida was created using a laser, 3) placed in isolation in a 15 ul droplet of G2 media, 4) incubated from day-3 to the blastocyst stage by day 5 or 6. Once the embryo was removed to undergo trophectoderm biopsy for PGS/CCS, 5) the spent media droplet was collected, frozen, then eventually tested for presence of DNA and genetic screening performed to compare to the corresponding trophectoderm biopsy result.

FIG. 2. Initial four samples tested with the highest levels of DNA after 2-hour whole genomic amplification. Only sample AB 16, outlined in red, had a DLRSD of <0.85, an indication of appropriate DNA quality and quantity to allow for an accurate genetic screening result. The DNA from this sample yielded a result of 45 KY, -13, identical to the blinded PGS result from the corresponding trophectoderm biopsy. After an additional hour of amplification, an additional sample yielded a DLRSD<0.085 and a result identical to trophectoderm biopsy. Overnight amplification did not produce improved sample accuracy in this cohort of samples.

### DETAILED DESCRIPTION OF THE INVENTION

The present invention is based on the discovery that free embryonic DNA can be prepared from spent IVF media. This discovery forms the basis for a new method for detecting genetic or chromosomal abnormalities in an embryo grown in vitro. This method can be used to improve outcomes for in vitro fertilization. Currently, PGS (preimplantation genetic screening) is a method where an embryo undergoes biopsy and is assessed for genetic normalcy prior to replacement into the uterine cavity. This is an expensive method and does not guarantee a live birth even in circumstances where an embryo deemed genetically normal is transferred into the uterine cavity. The minimally invasive approach of the invention confers a greater advantage over the traditional approach of PGS. The invention reduces risks to the embryo that can arise from conventional embryo biopsy. The methods of the invention provide an inexpensive, reliable, and non-invasive way to evaluate an embryo prior to placement into the uterine cavity.

Embryos have a predictable progression in vitro from a mature egg ready for fertilization to a blastocyst. Typically, a mature egg, retrieved on day 0, demonstrates evidence of fertilization by day 1 (2 pronuclei). By day 2, an embryo has divided into 2-6 cells called blastomeres. By day 3, an embryo divides into 4-10 blastomeres, with 8 being average for normal development. By day 4-5, an embryo becomes a morula, and by day 5-6 becomes a blastocyst. A blastocyst has an inner cell mass that is designated to become the fetus, and an outer set of cells called the trophectoderm, designated to become the placenta. As an embryo develops into a blastocyst, it releases various molecules into the growth media. The invention applies this discovery to methods for screening and detecting genetic or chromosomal abnormalities in embryos grown in vitro to reduce risk and improve outcomes for in vitro fertilization procedures.

Definitions

All scientific and technical terms used in this application have meanings commonly used in the art unless otherwise specified. As used in this application, the following words or phrases have the meanings specified.

As used herein, “reference sample” means any reference material known to be representative of normal genetic and/or chromosomal material.

As used herein, “a” or “an” means at least one, unless clearly indicated otherwise.

Methods for Preparing Embryonic DNA

In one embodiment, the invention provides a method of producing a preparation of embryonic DNA that can be performed without risking damage to a developing embryo. In one embodiment, the method comprises obtaining a sample of cell-free media in which an embryo was grown in vitro from the 4-10 blastomeres stage to the blastocyst stage of development, wherein the sample comprises cell-free embryonic DNA (cfeDNA). The method further comprises amplifying DNA obtained from the sample; and recovering the amplified cfeDNA. These steps can be carried out non-invasively, without performing a biopsy of the embryo. In one embodiment, the embryo is subjected to assisted hatching prior to obtaining the sample of media. In some embodiments, the sample of media is about 15-20 μl in volume. In some embodiments, the method further comprises analyzing copy number variations in the ploidy of the cfeDNA relative to a reference sample, or sequencing a target region of the DNA associated with a suspected genetic abnormality; and detecting the presence or absence of a genetic or chromosomal abnormality in the embryo. Representative examples of a chromosomal abnormality include, but are not limited to, a deletion or duplication of a chromosome. Representative examples of a genetic abnormality include, but are not limited to, a mutation associated with mortality or morbidity.

### Methods For Detecting Genetic or Chromosomal Abnormalities

In one embodiment, the invention provides a method of detecting a genetic or chromosomal abnormality in an embryo. Representative steps of the method comprise obtaining a sample of media in which the embryo was grown in vitro from the 4-10 blastomeres stage (about day 3 of development) to the blastocyst stage of development (about day 5 or 6 of development), wherein the sample comprises cell free embryonic DNA (cfeDNA). The method further comprises amplifying DNA obtained from the sample; and analyzing copy number variations in the ploidy of the sample relative to a reference sample, or sequencing a target region of the DNA associated with a suspected genetic abnormality. Variations in ploidy of the sample relative to the reference sample are indicative of a chromosomal abnormality, such as, for example, a deletion or duplication of a chromosome. Genetic abnormalities are indicated by variations in the DNA sequence associated with mortality or morbidity. The method thereby results in detecting a genetic or chromosomal abnormality in the embryo. In a typical embodiment, the embryo is subjected to assisted hatching prior to obtaining the sample of media. Assisted hatching means rupturing or dissolving the zona pellucida.

In one embodiment, the sample of media is about 20 μl in volume. The media sample can be from about 10 μl to about 40 μl. In some embodiments, the media sample is about 15-30 μl in volume. Those skilled in the art will appreciate the volume of media sample that is sufficient both to embryo growth in vitro, and to obtain sufficient cfeDNA, in view of the information provided in the working examples below.

In one embodiment, the analyzing comprises labeling and denaturing the amplified DNA, and hybridizing the labeled, denatured DNA in a 1:1 ratio to a normal metaphase spread of chromosomes.

In one embodiment, the analyzing comprises comparative genomic hybridization (CGH), fluorescence in situ hybridization (FISH), next generation gene sequencing, single nucleotide polymorphism microarrays (SNP), quantitative real time polymerase chain reaction (qPCR), and/or giemsa banding. In one embodiment, the CGH is array CGH (aCGH).

CGH is advantageous over the older and less comprehensive FISH. With microarray CGH, the actual DNA in the embryo is compared to a known normal DNA specimen (used as the reference sample) utilizing thousands of specific genetic markers. This gives a more accurate result, with far fewer false normal or false abnormal results.

Examples of a genetic abnormality involving a mutation associated with mortality or morbidity include, but are not limited to, common single gene disorders such as Cystic fibrosis, Tay-Sachs disease, Spinal muscular atrophy (SMA), Hemophilia, Sickle cell disease, Duchennes muscular dystrophy, and Thalassemia. Additional examples of genetic abnormalities include autosomal recessive disorders, such as Sanhoff disease, Gaucher disease, adenosine Deaminase deficiency, glycogen storage disease, Fanconi anemia, adrenal hyperplasia, phenylketonuria (PKU), and autosomal dominant disorders, such as Neurofibromatosis, Von-Hippel Lindau, myotonis dystrophy, Huntington's Disease, Marfan syndrome, osteogenesis imperfecta, Charcot-Marie-Tooth, APP early onset Alzheimers, polycystic kidney disease, retinitis pigmentosa, familial adenornatous polyposis, achondroplasia, and X-linked disorders, such as Ornithine carbamyl transferase deficiency, Fragile X, X-linked hydrocephalus.

Several studies have shown that overall about 50% of human preimplantation embryos from IVF are chromosomally abnormal. Chromosomes in eggs from older women have a significantly increased rate of abnormalities. Examples of a chromosomal abnormality, such as a deletion or duplication of a chromosome, include but are not limited to, trisomies, such as trisomy 13, 18, or 21, or aneuploidy involving sex chromosomes, such as Turner's syndrome and Klinefelter's syndrome, as well as aneuploidy involving other chromosomes. Chromosomal abnormalities can be responsible for failure of implantation of IVF embryos. Chromosomal abnormalities are also responsible for about 70% of miscarriages in early pregnancy.

Methods for Improving Outcome of In Vitro Fertilization

The invention additionally provides a method for optimizing the likelihood of a live birth after implantation of human egg fertilized in vitro. In one embodiment, the method comprises performing the method steps described above for detecting genetic or chromosomal abnormalities on a plurality of embryos developed after in vitro fertilization. The method further comprises selecting an embryo in which no genetic or chromosomal abnormality is detected. The method optionally comprises subsequently placing the selected embryo into the uterine cavity of a patient.

The invention further provides a method for reducing the risk of damage to a normal embryo that has been fertilized in vitro. The method comprises performing the method described above for detecting genetic or chromosomal abnormalities. The detecting is performed in lieu of trophectoderm biopsy on an embryo developed after in vitro fertilization. The method further comprises selecting an embryo in which no genetic or chromosomal abnormality is detected. The method optionally comprises subsequently placing the selected embryo into the uterine cavity of a patient.

### Kits

For use in the methods described herein, kits are also within the scope of the invention. Such kits can comprise a package or container that is compartmentalized to receive one or more containers such as vials, tubes, and the like, each of the container(s) comprising one of the separate elements (e.g., media, amplification reagents, labels, dyes) to be used in the method. The kit further comprises one or more containers, with one or more elements stored in the containers. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific application, and can also indicate directions for use. Directions and or other information can also be included on an insert which is included with the kit.

### EXAMPLES

The following examples are presented to illustrate the present invention and to assist one of ordinary skill in making and using the same. The examples are not intended in any way to otherwise limit the scope of the invention.

### Example 1: Free Embryonic DNA in Spent IVF Media

This Example demonstrates the presence of free embryonic DNA in spent media, with a PGS result concordant with trophectoderm biopsy. A prospective cohort analysis was used to assess if preimplantation genetic screening (PGS) is possible by testing for free embryonic DNA in spent IVF media from embryos undergoing trophectoderm biopsy. The study involved seven patients undergoing IVF in an academic fertility center with 57 embryos undergoing trophectoderm biopsy for PGS. On day 3 of development, each embryo was placed in a separate media droplet. All biopsied embryos received a PGS result via array comparative genomic hybridization (aCGH). PGS was performed on amplified DNA extracted from media and results were compared with PGS results for the corresponding biopsy. The main outcome measures included: 1) presence of DNA in spent IVF culture media, and correlation between genetic screening result from spent media and corresponding biopsy. Fifty five samples had detectable DNA ranging from 2 to 642 ng/μl after 2 hour amplification. Six samples with the highest DNA levels underwent PGS, rendering one result with a Derivative Log Ratio Standard Deviation (DLRSD)<0.85 (a quality control metric of oligonucleotide array CGH). The fluid sample and trophectoderm results were identical demonstrating (45XY, −13). Three samples were re-amplified one hour and tested showing improving DLRSD. One of the three samples with a DLRSD of 0.85 demonstrated (46XY), consistent with biopsy. Overnight DNA amplification showed DNA in all samples. This Example thus demonstrates two novel findings: the presence of free embryonic DNA in spent media, and that a result can be rendered which is consistent with trophectoderm biopsy. Via methods of DNA collection, amplification, and testing described herein, PGS can be performed without biopsy.

Although in recent years dramatic improvements in IVF success rates have been achieved, reproductive medicine remains fraught with inefficiency pertaining to risk of multiple pregnancy and miscarriage (1-3). Ideally, transferring one euploid embryo at a time would decrease the risk of both of these serious complications while simultaneously increasing the precision and efficiency of in vitro fertilization (IVF) (4-5). One would therefore presume that preimplantation genetic screening (PGS) of the embryo would address goals for increased precision and risk reduction (6).

Biopsy, an invasive technique, currently remains the only reliable approach to obtain sufficient embryonic deoxyribonucleic acid (DNA) for genetic screening. A screening technique for embryonic competency without the need for biopsy would avoid any potential risk due to an invasive procedure and would thus be an important advancement in assisted reproduction. To date, there have been several approaches to evaluate embryos in the laboratory to determine embryonic viability without biopsy. These include morphologic assessment based on various characteristics noted at differential time points of embryonic development (7-8), time-lapse imaging (9-11), assessment of DNA from the blastocele fluid (12), and assessment of the “secretome” within the spent IVF media, specifically metabolomics (13-16), proteomics (17-19), and analysis of micro-RNAs (20-22). None of these approaches have been reliable in accurately determining if an embryo, once transferred, will undergo sustained implantation and lead to a healthy live birth.

Within the last five years, non-invasive prenatal screening (NIPT) has revolutionized the way obstetricians evaluate pregnant patients during the late first and early second trimester of pregnancy, demonstrating high sensitivities, specificities, and negative predictive values for detection of the most common aneuploidies (23-32). This method utilizes techniques to distinguish and assess free fetal DNA from that of maternal origin from a single maternal blood draw obtained at 10 weeks gestation or beyond. Initially, this approach was recommended for patients at high risk for fetal aneuploidy (33-35); however, a recent prospective randomized trial demonstrated benefit for the general population (32). Free fetal DNA exists in maternal blood due to cell turnover as the placenta and fetus develop. It is unknown when free DNA is released, whether that starts as soon as an embryo begins to divide, or at a later time point in embryonic development.

The present study was prompted by a hypothesis that free embryonic DNA is released early in embryonic development, soon after an embryo begins to divide. As such, the study described in this Example was designed to demonstrate that free embryonic DNA is present in spent IVF culture media. To date, there have been no peer-reviewed studies demonstrating presence of free embryonic DNA in spent media in which an embryo has developed. Furthermore, the Example demonstrates that a genetic screening result can be rendered similar to that of the corresponding trophectoderm biopsy.

### Materials and Methods

Study Subjects

Institutional Review Board approval was obtained, and participation for enrollment was offered to patients undergoing IVF with trophectoderm biopsy for PGS/Comprehensive Chromosomal Screening (CCS), at the University of California, Los Angeles. There were no exclusion criteria, Study subjects agreed to additional testing of the spent media with the understanding that this additional testing would not have any impact on fertility treatment timeline or outcome. There was no added patient time nor expense for study participation and no known effect on pregnancy outcomes.

Preparation and Collection of Spent Media

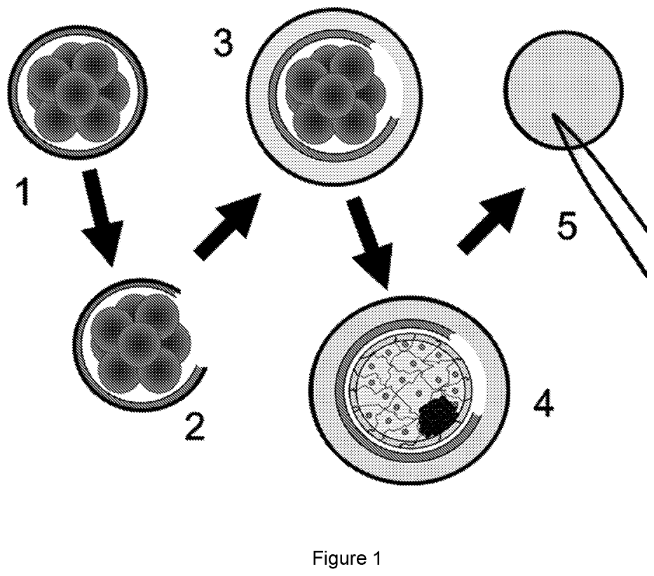

On day 3 of embryo development, each embryo was separated from the G1 media in which it was developing with up to five embryos, and was placed in a separate single 15 ul droplet of G2 media and assisted hatching was performed. The purpose of assisted hatching was two-fold: to allow for extrusion of trophectoderm by day-5/6 to facilitate trophectoderm biopsy, and to allow expulsion of free embryonic DNA, if present, through the opening created by hatching into the G2 media, Assisted hatching on day-3 of embryonic development is standard operating procedure in the laboratory for embryos intended for PGS. On day 5 or 6 of development, for each blastocyst that qualified for trophectoderm biopsy based on laboratory criteria (expanding blastocyst with clear distinction between the inner cell mass and trophectoderm), the spent media in which it developed was collected in a sterile vial and frozen at −80 degrees F. Refer to FIG. 1 for a visual representation of the aforementioned steps. All qualifying embryos then underwent trophectoderm biopsy, frozen via vitrification, and received a PGS result utilizing aCGH and embryo transfer was performed as planned. Spent media was frozen when collected, then evaluated by a separate genetic screening laboratory blinded to trophectoderm biopsy CCS results, as noted below.

Evaluation of media for DNA

A separate College of American Pathologists-accredited PGS laboratory blinded to any patient characteristics and trophectoderm biopsy results assessed all media used in the laboratory for incubation of egg, sperm, or embryo for DNA contamination. These media were all commercially manufactured, and included 1) Oocyte Retrieval Flush Media: Modified HTF Medium (Human Tubal Fluid HEPES Buffered with Gentamicin) (Irvine Scientific), 2) Oocyte Retrieval Media for washing oocytes during retrieval: G-MOPS (Vitrolife) supplemented with Serum Substitute Supplement (SSS) (Irvine Scientific), 3) Medias used during Sperm Processing: Gradient: Isolate (Sperm Separation Medium) (Irvine Scientific) and Wash: Sperm Rinse (Vitrolife), 4) Oil Overlay for Culture: OVOIL (Vitrolife), 5) Culture Media (Vitrolife): Day 0: GIVF, Day 1 to 3: G.1 Plus, Day 3 to 6: G.2 Plus (Vitrolife), 6) ICSI Media: G-MOPS (Vitrolife) supplemented with Serum Substitute Supplement (SSS) (Irvine Scientific), 7) Biopsy Media: Day 3 Biopsy (tested for contamination but not used in this study): G-PGD (Vitrolife) Supplemented with Human Serum Albumin (HSA) (Irvine Scientific), Day 5/6 Biopsy: G-MOPS (Vitrolife) supplemented with Serum Substitute Supplement (SSS) for (Irvine Scientific), and Transfer Media: EmbryoGlue (Vitrolife).

After retrieval, the oocytes were collected in G-Mops Media (Vitrolife) supplemented 10% with Serum Substitute Supplement (Irvine Scientific). All mature oocytes (MIl) were fertilized via intracytoplasmic sperm injection (ICSI) and grown for one day (day 0-1) in G IVF Plus Media (Vitrolife). The embryos were then switched on day 1 to G 1.5 Plus G1 Media (Vitrolife) and grown from day 1-3. On day 3, the embryos underwent assisted hatching and were then transferred to 2.5 Plus G2 Media (Vitrolife) and cultured from day-3 to day-5/6. The spent G2 media was collected for further evaluation for DNA. Each spent media droplet that was collected on day 5 or 6 for embryos undergoing trophectoderm biopsy was processed for DNA amplification (see DNA Amplification Method below). Comprehensive chromosomal screening was performed on the media via aCGH of droplets with the highest DNA content and the result was compared with the PGS result obtained for the corresponding embryo biopsy.

Whole Genomic Amplification (WGA) and Screening Methods for Spent Media

Samples were amplified for 2 hours using Repli-G single cell kit (Qiagen), and quantified by Qubit (broad range DNA assay kit). After amplification, DNA was labeled, purified and hybridized for array CGH performed overnight. Samples were then re-amplified overnight to assess change in DNA quantity per sample. Amplified DNA was assessed for chromosome number via a previously validated oligonucleotide aCGH (Agilent Technologies) (36). In order for a sample reading to be considered reliable, the Derivative Log Ratio Standard Deviation (DLRSD), a company internal quality control metric indicator of confidence and reliability of the sample for oligonucleotide aCGH, should be <0.85 (Agilent Technologies). The DLRSD is a measure of the 1 sigma error of the log ratios on an array. It is computed from the interquartile range of differences of log ratios reported for probes sequentially ordered along the genome. A DLRSD score >0.85 reflects high probe-to-probe log ratio noise. A DLRSD result above this number was considered unreliable and therefore an accurate screening result could not be rendered.

Statistical Analyses

All statistical analyses were performed using STATA software (STATA/SE 14.0). Chi-Square was utilized to assess categorical outcomes, and two-sided t-test was utilized to compare means.

### Results

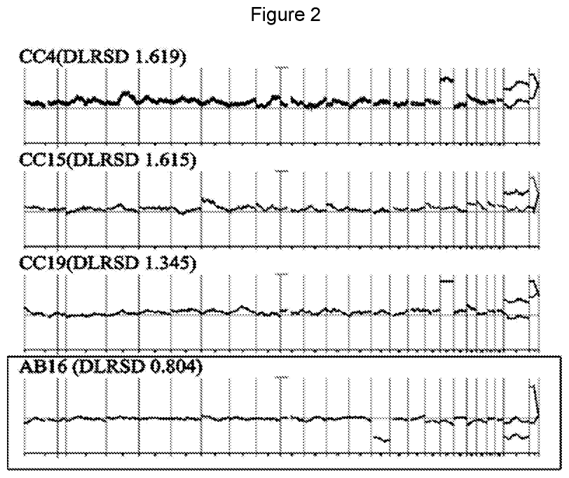

Seven patients ranging in age from 25 to 42 years and male partners ranging in age from 31 to 54 years participated in the study, resulting in 57 embryos undergoing trophectoderm biopsy and 57 corresponding samples of spent media collected. Prior to testing the spent media, all types of media used in the laboratory in which sperm, egg, or embryo were to be exposed, were amplified to be examined for DNA as a contaminant, and no DNA was found. Next, the 57 samples were amplified for 2 hours and tested for the presence of DNA. Fifty five of the 57 samples had detectable DNA ranging from 2 to 642 ng/ul (Table 1). Six samples with the highest quantities ranging in DNA levels from 52 to 642 ng/ul underwent aCGH, rendering one result with DLRSD <0.85 in the sample with 642 ng/ul. The fluid sample and trophectoderm results were identical demonstrating (45XY, −13) (FIG. 2). The remaining samples had an unreliable DLRSD >0.85. Three samples with the highest level of DNA were re-amplified for an additional hour and screened showing improving DLRSD, however levels remained >0.85 with one exception. One of the three samples with the lowest DLRSD at 0.85 demonstrated a 46XY embryo, consistent with the trophectoderm biopsy. Remaining samples were then amplified overnight. All samples demonstrated DNA at much higher concentrations. However, aCGH of samples with the highest DNA content all revealed DLRSD well above 0.85 thus preventing a reliable screening result.

### Discussion

Currently, trophectoderm biopsy with CCS is the standard of care with regard to genetic screening of embryos prior to implantation and is highly reproducible between clinics (37). Embryonic genetic assessment has evolved over the past 2 decades. Initially, blastomere biopsy on day-3 of development with florescence in-situ hybridization (FISH) of two to five chromosomes was utilized (38-40); however, several prospective randomized trials evaluating day-3 or trophectoderm biopsy with FISH demonstrated either no improvement or a detriment in live birth outcomes (41-49). A systematic review and meta-analysis of nine prospective randomized controlled trials confirmed a detrimental impact of this approach (50). Hypotheses for poorer outcomes included poor reliability and accuracy by FISH and possible damage secondary to the biopsy itself. This led to a shift from day-3 biopsy to that of the trophectoderm.

Despite the fact that more cells are removed with trophectoderm biopsy, a smaller percentage of the embryo is removed as compared to day-3, and no cells are disrupted in the inner cell mass. The safety of day-5/6 trophectoderm biopsy and the harm of day-3 biopsy were demonstrated in a blinded prospective randomized trial (51). Yet the presumption of safety may not be applicable to laboratories with less experience in biopsy technique.

Notably, randomized controlled trials have demonstrated benefit with trophectoderm biopsy when compared with non-biopsied controls, providing similar pregnancy rates when transferring one tested embryo vs, two untested embryos, while virtually eliminating multiple pregnancy in the test group but not in controls (52). Furthermore, improvements in whole chromosomic evaluation have developed which offer tremendous advantages over FISH. These methods including aCGH (53), single nucleotide polymorphism (SNP-) microarray (54), real-time quantitative polymerase chain reaction (qPCR) (55), and most recently next generation gene sequencing (NGS) (56). Each of these techniques offers a high degree of reliability and reproducibility.

For the first time, this Example demonstrates presence of free embryonic DNA in spent IVF media collected from embryos grown from day-3 cleavage stage to a day-5/6 blastocyst. All samples demonstrated free DNA at some level, and two samples were of high enough quality and quantity for genetic screening, yielding a result identical to the corresponding trophectoderm biopsy. The prospect of genetic screening of an embryo in vitro without biopsy is appealing, as it eliminates any risk of damage from an invasive procedure such as embryo biopsy. In order for embryonic genetic screening of spent IVF media to become more reliable, several steps in the process can be optimized. These include improving DNA collection methods, DNA amplification, and screening techniques.

The data presented herein demonstrated that free embryonic DNA is released very early in embryonic development, at a minimum from day-5 of development and likely as early as day-3. Media were collected from day-3 to day-5/6 of development for two reasons: 1) this was a convenient time point in the laboratory assessment of embryos where embryos are transported from the sequential G1 media (formulated for days 1 and 2 of embryonic development) into G2 media (optimized for day-3 cleavage stage to blastocyst formation), and 2) assisted hatching is performed in order to facilitate biopsy by the blastocyst stage, as trophectoderm cells often herniate through the opening and are therefore more easily accessed and removed for testing. It was hypothesized that an opening in the zona pellucida will lead to better extrusion of DNA from the opening into the media, though the study did not test to see if unhatched embryos excreted DNA into the media to a similar level or at all. There is no evidence of harm or a disadvantage of assisted hatching on day-3 as opposed to waiting to the day of blastocyst biopsy. The media in which embryos were exposed from the first 3 days of development were not tested. Now that monophasic media exists allowing an embryo to reliably grow from day-1 to a blastocyst (57-58), the two days of added exposure may lead to a higher level of DNA excreted into the media, and therefore merits evaluation, Alternatively, in order to minimize DNA degradation, the best approach may be to collect the media daily and freeze it until all media samples can be combined, amplified, and tested. These approaches may lead to higher levels and quality of DNA for assessment.

WGA was performed using RepliG, a technique utilizing Phi 29 polymerase and multiple displacement amplification (MDA) that is shown to cover up to 72% of the genome (59). This method covers a greater percentage of the genome than PCR-based available WGA methods estimated to cover up to 36% of the genome (60). It was hypothesized that greater coverage of the genome will lead to higher quality and quantity of the amplified DNA product. However, these techniques are prone to amplification bias: PCR-based methods can have sequence-dependent bias due to exponential amplification of random primers (61), and multiple displacement amplification can have amplification bias due to non-linear amplification (62). Despite the fact that MDA offers greater genome coverage than PCR, PCR techniques may harbor greater accuracy with regard to DNA copy number analysis (63), and thus merits further assessment. Other DNA amplification methods exist and warrant evaluation. This includes Multiple Annealing and Looping Based Amplification Cycles (MALBAC) which is estimated to cover up to 93% of the genome with low allelic drop out (59). Those skilled in the art will appreciate other approaches that can be examined to optimize the WGA method.

The accuracy of DNA screening methods has evolved dramatically over the past 5 years. Debate exists as to whether aCGH, SNP-microarray, and qPCR offer advantages over the other, but NGS may offer the greatest accuracy and reliability. The main drawback with NGS is that it currently takes 7-10 days from biopsy to render a screening result, unlike aCGH and qPCR that can yield a result in less than 24 hours. The media were not assessed with this approach as NGS has just recently been validated for CCS, though NGS may still require similar high-quality DNA amplification in order to yield a reliable screening result. The present study used oligonucleotide aCGH, which allows for far greater chromosome region coverage than other currently available aCGH and qPCR technologies (36). Nevertheless, NGS, with its high degree of accuracy and reliability, could be tested for performance.

The greatest advantage of embryonic screening of spent IVF media would be the ability to evaluate an embryo without the potential for damage from biopsy. This benefit makes this approach more attractive, leading to greater and potentially global utilization by patients. However, there may be some disadvantages. Currently, the majority of IVF laboratories develop several embryos in one media droplet, generally five embryos in each. If assessment of spent media were to become viable, this would require placing only one embryo per droplet. Consequently, a greater amount of IVF media, and a larger number of Petri dishes to house the droplets, would be required. This in turn would lead to a greater number of incubators and require a larger amount of embryology laboratory space, time and effort. Though all embryos would undergo assisted hatching, one can argue that cost will go down due to the lack of an embryo biopsy charge, however the increased material necessary may offset this cost benefit.

There are several strengths attributable to the findings presented herein. Unused media of all types utilized in the IVF laboratory setting do not have identifiable DNA. The data presented herein further show that media exposed to developing embryos acquires free DNA and that a genetic screening result can be rendered similar to the corresponding trophectoderm biopsy. Even though this technique shows tremendous promise, however, there are still no data demonstrating that PGS improves live birth rate on a per retrieval basis for all patient populations (64). The number of samples tested thus far is small. The vast majority of samples yielded a DLRSD above 0.85 demonstrating quantity and/or quality insufficient to reach a reliable screening result; and thus only 2 samples were of reliable quality to yield a screening result, albeit both were identical to the corresponding biopsy.

### CONCLUSION

This Example demonstrates that embryonic screening of spent IVF media is possible in the IVF setting. The results reveal that free DNA exists in the spent media and can render a result similar to trophectoderm biopsy.

### REFERENCES

1. Centers for Disease Control and Prevention, American Society for Reproductive Medicine, Society for Assisted Reproductive Technology. 2009 Assisted Reproductive Technology Success Rates: National Summary and Fertility Clinic Reports. Atlanta: US Department of Health and Human Services; 2011.

2. Sunderum S, Kissin D M, Crawford S B, Folger S G, Jamieson D J, Warner L, et al. Assisted Reproductive Technology Surveillance—United States, 2013. MMWR Surveill Summ 2015; 64:1-25.

3. Carpinello O J, Casson P R, Kuo C L, Raj R S, Sills E S, Jones C A. Cost implications for Subsequent Perinatal Outcomes After IVF Stratified by Number of Embryos Transferred: A Five Year Analysis of Vermont Data. Appl Health Econ Health Policy 2016; March 11 Epub ahead of print.

4. Forman E J, Hong K H, Franasiak J M, Scott R T Jr. Obstetrical and neonatal outcomes form the BEST Trial: single embryo transfer with aneuploidy screening improves outcomes after in vitro fertilization without compromising fertility rates. Am J Obstet Gyencol 2014; 210:157.e1-6.

5. La Sala G B, Morini D, Gizzo S, Nicoll A, Palomba S. Two consecutive single pregnancies versus one twins pregnancy as preferred outcome of in vitro fertilization for mothers and infants: a retrospective case-control study. Curr Med Res Opin 2016; 32:687-92.

6. Styer A K, Luke B, Vitek W, Chistianson M S, Baker V L, Christy A Y, et al. Factors associated with the use of elective single-embryo transfer and pregnancy outcomes in the United States, 2004-2012. Fertil Steril 2016; March 18 Epub ahead of print.

7. Lazzaroni-Tealdi E, Barad D H, Albertini D F, Yu Y, Kushnir V A, Russell H, et al. Oocyte Scoring Enhances Embryo-Scoring in Predicting Pregnancy Chances with IVF Where It Counts Most. PLoS One 2015; 10:e0143632.

8. Yin H, Jiang H, He R, Wang C, Zhu J, Li Y. The effects of blastocyst morphological score and blastocoele re-expansion speed after warming on pregnancy outcomes, Clin Exp Reprod Med 2016; 43:31-7.

9. Adamson G D, Abusief M E, Palao L, Witmer J, Palao L M, Gvakharia M. Improved implantation rates of day 3 embryo transfers with the use of an automated time-lapse-enabled test to aid in the embryo selection. Fertil Steril 2016; 105:369-75.

10. Liu Y, Chapple V, Feenan K, Roberts P, Matson P. Time-lapse deselection model for human day 3 in vitro fertilization embryos: the combination of qualitative and quantitative measures of human growth. Fertil Steril 2016; 105:656-62.

11. Motato Y, de Los Santos M J, Escriba M J, Ruiz B A, Remohi J, Mesequer M. Morphokinetic analysis and embryonic prediction for blastocyst formation through an integrated time-lapse system. Fertility Steril 2016; 105:376-84.

12. Gianaroli L, Magli M C, Pomante A, Crivello A M, Cafueri G, Valerio M, et al. Blastocentesis: a source of DNA for preimplantation genetic testing. Results from a pilot study. Fertil Steril 2014; 102:1692-9.

13. Uyar A, Seli E. Metabolomic assessment of embryo viability. Semin Reprod Med 2014; 32:141-52.

14. Sakkas D. Embryo selection using metabolomics. Methods Mol Bio 2014; 1154:533-40.

15. Bellver J, De Los Santos M J, Alama P, Castello D, Privitera L, Galliano D, et al. Day-3 embryo metabolomics in the spent culture media is altered in obese women undergoing in vitro fertilization. Fertil Steril 2015; 103:1407-15.

16. Rødgaard T, Heegaard P M, Callesen H. Non-invasive assessment of in-vitro embryo quality to improve transfer success. Reprod Biomed Online 2015; 31:585-92.

17. Katz-Jaffe M G, McReynolds S, Gardner D K, Schoolcraft W B. The role of proteomics in defining the human embryonic secretome. Mol Hum Reprod 2009; 15;271-7.

18. Nyalwidhe J, Burch T, Bocca S, Cazares L, Green-Mitchell S, Cooke M, et al. The search for biomarkers of human embryo developmental potential in IVF: a comprehensive proteomic approach. Mol Hum Reprod 2013; 19:250-63.

19. Benkhalifa M, Madkour A, Louanjli N, Bouamoud N, Saadani B, Kaarouch I, et al. From global proteome profiling to single targeted molecules of follicular fluid and oocyte: contribution to embryo development and IVF outcome. Expert Rev Proteomics 2015; 12:407-23.

20. Cuman C, Van Sinderen M, Gantier M P, Rainczuk K, Sorby K, Rombauts L, et al. Human blastocyst secreted microRNA regulate endometrial epithelial cell adhesion, EBioMedicine 2015; 11:1528-35.

21. Kropp J, Khatib H. mRNA fragments in in vitro culture media are associated with bovine preimplantation embryonic development. Front Genet 2015; 24:273.

22. Rosenbluth E M, Shelton D N, Sparks A E, Devor E, Christenson L, Van Voorhis B J. MicroRNA expression in the human blastocyst. Fertil Steril 2013; 99:855-61.

23. Chiu R W, Akolekar R, Zheng Y W, Leung T Y, Sun H, Chan K C, et al. Non-invasive prenatal assessment of trisomy 21 by multiplexed maternal plasma DNA sequencing: large scale validity study. BMJ 2011; 342:c7401.

24. Ehrich M, Deciu C, Zwiefelhofer T, Tynan J A, Cagasan L, Tim R, et al. Noninvasive detection of fetal trisomy 21 by sequencing of DNA in maternal blood: a study in a clinical setting. Am J Obstet Gynecol 2011: 204:205.e1-205.

25. Sehnert A J, Rhees B, Comstock D, de Feo E, Heilek G, Burke J, et al. Optimal detection of fetal chromosomal abnormalities by massively parallel DNA sequencing of cell-free fetal DNA from maternal blood, Clin Chem 2011; 57:1042-9.

26. Palomaki G E, Kloza E M, Lambert-Messerlian G M, Haddow J E, Neveax L M, Ehrich M, et al. DNA sequencing of maternal plasma to detect Down syndrome: an international clinical validation study. Genet Med 2011; 13:913-20.

27. Sparks A B, Wang E T, Struble C A, Barrett W, Stokowski R, McBride C, et al. Selective analysis of cell-free DNA in maternal blood for evaluation of fetal trisomy. Prenat Diagn 2012; 32:3-9.

28. Palomaki G E, Deciu C, Kloza E M, Lambert-Messerlian G M, Haddow J E, Neveax L M, et al. DNA sequencing of maternal plasma reliably identifies trisomy 18 and trisomy 13 as well as Down Syndrome: an international collaborative study. Genet Med 2012; 14:296-305.

29. Bianchi D W, Platt L D, Goldberg J D, Abuhamad A Z, Sehnert A J, Rava R P, et al. Genome-wide fetal aneuploidy detection by maternal plasma DNA sequencing. Obstet Gynecol 2012; 119:890-901.

30. Norton M E, Brar H, Weiss J, Karimi A, Laurent L C, Caughey A B, et al, Non-Invasive Chromosomal Evaluation (NICE) Study: results of a multicenter prospective cohort study for detection of fetal trisomy 21 and trisomy 18. Am J Obstet Gynecol 2012; 207:137.e1-137.e8.

31. Dan S, Wang W, Ren J, Li Y, Hu H, Xu Z, et al, Clinical application of massively parallel sequencing-based prenatal noninvasive fetal trisomy test for trisomies 21 and 18 in 11,105 pregnancies with mixed risk factors. Prenat Diagn 2012; 32:1225-32.

32. Bianchi D W, Parker R L, Wentworth J, Madankumar R, Saffer C, Das A F, et al. DNA sequencing versus standard prenatal aneuploidy screening. N Eng J Med 2014; 370:799-808.

33. Benn P, Borell A, Chiu R, Cuckle H, Dugoff L, Faas B, et al. Position statement from the Aneuploidy Screening Committee on behalf of the Board of the International Society for Prenatal Diagnosis. Prenat Diagn 2013; 33:622-9.

34. Devers P L, Cronister A, Ormond K E, Facio F, Brasington C K, Flodman P. Noninvasive prenatal testing/noninvasive prenatal diagnosis: the position of the National Society of Genetic Counselors. J Genet Couns 2013; 22:291-5.

35. American College of Obstetricians Gynecologists Committee on Genetics. Committee Opinion No. 545: noninvasive prenatal testing for fetal aneuploidy. Obstet Gynecol 2012; 120:1532-4.

36. He W, Sun X, Liu L, Li M, Jin H, Wang W H. The prevalence of chromosomal deletions relating to developmental delay and/or intellectual disability in human euploid blastocysts. PLOS One 2014:9:e85207,

37. Capalbo A, Ubaldi F M, Cimadomo D, Maggiulli R, Patassini C, Dusi L, et al. Consistent and reproducible outcomes of blastocyst biopsy and aneuploidy screening across different biopsy practitioners: a multicenter study involving 2586 embryo biopsies. Hum Reprod 2016; 13:199-208.

38. Delhanty J D, Griffen D K, Handyside A H, Harper J, Atkinson G H, Pieters M H, et al. Detection of aneuploidy and chromosomal mosaicism in human embryos during preimplantation sex determination by florescent in-situ hybridization (FISH). Hum Mol Genet 1993; 2:1183-5.

39. Munne S, Lee A, Rosenwaks Z, Grifo J, Cohen J. Diagnosis of major chromosome aneuploides in human preimplantation embryos. Hum Reprod 1993; 8:2185-91.

40. Verlinsky Y, Cieslak J, Freidine M, Ivakhnenko V, Wolf G, Kovalinskaya L, et al. Pregnancies following pre-conception diagnosis of common aneuploidies by florescence in-situ hybridization. Hum Reprod 1995; 10:1923-7.

41. Staessen C, Platteau P, Van Assche E, Michiels A, Tournaye H, Camus M, et al. A comparison of blastocyst transfer with or without preimplantation genetic diagnosis for aneuploidy screening in couples with advanced maternal age: a prospective randomized controlled trial. Hum Reprod 2004; 19:2849-58.

42. Mastenbroek S, Twisk M, van Echten-Arends J, Sikkema-Raddatz B, Korevaar J C, Verhoeve H R, et al. In vitro fertilization with preimplantation genetic screening. N Eng J Med 2007; 357:9-17.

43. Blockeel C, Schutyser V, De Vos A, Verpoest W, De Vos M, Staessen C, et al. Prospectively randomized controlled trial of PGS in IVF/ICSI patients with poor implantation, Reprod Biomed Online 2008; 17:848-54.

44. Hardarson T, Hanson C, Lundin K, Hillensjö T, Nilsson L, Stevic J, et al. Preimplantation genetic screening in women of advanced maternal age caused a decrease in clinical pregnancy rate: a randomized controlled trial. Hum Reprod 2008; 23:2806-12.

45. Jansen R P, Bowman M C, de Boer K A, Leigh D A, Lieberman D B, McArthur S J. What next for preimplantation genetic screening (PGS)? Experience with blastocyst biopsy and testing for aneuploidy. Hum Reprod 2008; 23:1476-8.

46. Staessen C, Verpoest W, Donoso P, Haentjens P, Van der Elst J, Liebaers I, et al. Preimplantation genetic screening does not improve delivery rate in women under the age of 36 following single-embryo transfer. Hum Reprod 2008; 23:2818-25.

47. Debrock S, Melotte C, Spiessens C, Peeraer K, Vanneste E, Meeuwis L, et al. Preimplantation genetic screening for aneuploidy of embryos after in vitro fertilization in women aged at least 35 years: a prospective randomized trial, Fertil Steril 2010; 93:364-73.

48. Meyer L R, Klipstein S, Hazzlet W D, Nasta T, Mangan P, Karande V C. A prospective randomized controlled trial of preimplantation genetic screening in the ‘good prognosis’ patient. Fertil Steril 2009; 91:1731-8.

49. Schoolcraft W B, Katz-Jaffe M G, Stevens J, Rawlins M, Munne S. Preimplantation aneuploidy testing for infertile patients of advanced maternal age: a randomized prospective trial. Fertil Steril 2009; 92:157-62.

50. Mastenbroek S, Twisk M, van der Veen F, Repping S. Preimplantation genetic screening: a systematic review and meta-analysis of RCTs. Hum Reprod Update 2011; 17:454-66.

51. Scott R T, Upham K M, Forman E J, Zhao T, Treff N R. Cleavage-stage biopsy significantly impairs human embryonic implantation potential while blastocyst biopsy does not: a randomized and paired clinical trial. Fertil Steril 2013:100:624-30,

52. Forman E J, Hong K H, Ferry K M, Tao X, Taylor O, Levy B, et al. In vitro fertilization with a single euploid blastocyst transfer: a randomized controlled trial. Fertil Steril 2013; 100:100-7.

53. Harper J C, Harton G. The use of arrays in preimplantation genetic diagnosis and screening. Fertil Steril 2010; 94:1173-7,

54. Tobler K J, Brezina P R, Benner A T, Du L, Xu X, Kearns W G. Two different microarray technologies for preimplantation genetic diagnosis and screening, due to reciprocal translocation imbalances, demonstrate equivalent euploidy and clinical pregnancy rates. J Assist Reprod Genet 2014; 31:843-50.

55. Treff N R, Tao X, Ferry K M, Su J, Taylor D, Scott R T Jr. Development and validation of an accurate quantitative real-time polymerase chain reaction-based assay for human blastocyst comprehensive chromosomal aneuploidy screening, Fertil Steril 2012; 97:819-24.

56. Zheng H, Jin H, Liu L, Liu J, Wang W H. Application of next-generation sequencing for 24-chromosome aneuploidy screening of human preimplantation embryos. Mol Cytogenet 2015; 8:38.

57. Hardarson T, Bungum M, Conaghan J, Meintjes M, Chantilis S J, Molnar L, et al. Noninferiority, randomized, controlled trial comparing embryo development using media developed for sequential or undisturbed culture in a time-lapse set up. Fertil Steril 2015; 140:1452-9.

58. Werner M D, Hong K H, Franasiak J M, Forman E J, Reda C V, Molinaro T A, et al, Sequential versus Monophasic Media Impact Trial (SuMMIT): a paired randomized controlled trial comparing a sequential media system to a monophasic medium. Fertil Seril 2016; Jan. 27 Epub ahead of print.

59. Zong C, Lu S, Chapman A R, Xie X S. Genome-wide detection of single-nucleotide and copy-number variations of a single human cell. Science 2012; 338:1622-6,

60. Voet T, Kumar P, Van Loo P, Cooke S L, Marshall J, Lin M L, et al. Single-cell paired-end genome sequencing reveals structural variation per cell cycle. Nucleic Acids Res 2013; 41:6119-38.

61. Telenius H, Carter N P, Bebb C E, Nordenskjold M, Ponder B A, Tunnacliffe A. Degenerate oligonucleotide-primed PCR: general amplification of target DNA by a single degenerate primer. Genomics 1992; 13:718.

62. Dean F B, Nelson, J R, Giesler T L, Lasken R S. Rapid amplification of plasmid and phage DNA using Phi 29 DNA polymerase and multiply-primed rolling circle amplification. Genome Res 2001; 11:1095,

63. Treff N R, Su J, Tao X, Northrop L E, Scott R T Jr. Single-cell whole-genome amplification technique impacts the accuracy of SNP microarray-based genotyping and copy number analyses. Mol Hum Reprod 2011; 17:335-43.

64. Mastenbroek S, Repping S. Preimplantation genetic screening: back to the future, Hum Reprod 2014; 29:1846-50.

Example 2: Extended Amplification of Free Embryonic DNA From Embryo Culture Media

The procedure described above was modified to improve DNA yield by extending the amplification of the samples. The samples were amplified overnight, which was successful in that it yielded much higher levels of DNA. Screening was then performed on two samples with the highest levels of DNA. Reliability of the diagnosis was reduced, however, due to DNA quality. These results suggest extending amplification is a useful strategy for increasing DNA yield, but should be accompanied by additional measures to preserve DNA quality. This can be accomplished through adjustment of amplification conditions and/or by limiting the extended amplification period to less than overnight. Those skilled in the art are aware of adjustments that can be made to optimize amplification conditions, such as, for example, number of cycles, extension time, annealing time and temperature, denaturation time and temperature, dNTP concentration, polymerase concentration, magnesium concentration, and primer conditions.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method of producing a preparation of embryonic DNA, the method comprising:
a) obtaining a sample of cell-free media in which an embryo was grown in vitro from the 4-10 blastomeres stage to the blastocyst stage of development, wherein the sample comprises cell-free embryonic DNA (cfeDNA);
b) amplifying DNA obtained from the sample; and
c) recovering the amplified cfeDNA without performing a biopsy of the embryo.

2. The method of claim 1, wherein the embryo is subjected to assisted hatching prior to obtaining the sample of media.

3. The method of claim 4, wherein the sample of media is about 15-20 μl in volume.

4. The method of claim 1, further comprising:
a) analyzing copy number variations in the ploidy of the cfeDNA relative to a reference sample or sequencing a target region of the DNA associated with a suspected genetic abnormality; and
b) detecting the presence or absence of a genetic or chromosomal abnormality in the embryo.

5. The method of claim 4, wherein the chromosomal abnormality is a deletion or duplication of a chromosome.

6. The method of claim 4, wherein the genetic abnormality is a mutation associated with mortality or morbidity.

7. The method of claim 4, wherein the analyzing comprises labeling and denaturing the amplified DNA, and hybridizing the labeled, denatured DNA in a 1:1 ratio to a normal metaphase spread of chromosomes.

8. The method of claim 4, wherein the analyzing comprises comparative genomic hybridization (CGH), fluorescence in situ hybridization (FISH), next generation gene sequencing, and/or giemsa banding.

9. The method of claim 8, wherein the CGH is array CGH.

10. A method for optimizing the likelihood of a live birth after implantation of human egg fertilized in vitro comprising performing the method of claim 4 on a plurality of embryos developed after in vitro fertilization and selecting an embryo in which no genetic or chromosomal abnormality is detected.

11. The method of claim 10, wherein the abnormality is a deletion or duplication of a chromosome, or a mutation associated with mortality or morbidity.

12. The method of claim 10, wherein the analyzing comprises labeling and denaturing the amplified DNA, and hybridizing the labeled, denatured DNA in a 1:1 ratio to a normal metaphase spread of chromosomes.

13. The method of claim 10, wherein the analyzing comprises comparative genomic hybridization (CGH), fluorescence in situ hybridization (FISH), next generation gene sequencing, and/or giemsa banding.

14. The method of claim 13, wherein the CGH is array CGH.

15. A method for reducing the risk of damage to a normal embryo that has been fertilized in vitro comprising performing the method of claim 4 in lieu of trophectoderm biopsy on an embryo developed after in vitro fertilization.

16. The method of claim 15, wherein the abnormality is a deletion or duplication of a chromosome, or a mutation associated with mortality or morbidity.

17. The method of claim 15, wherein the analyzing comprises labeling and denaturing the amplified DNA, and hybridizing the labeled, denatured DNA in a 1:1 ratio to a normal metaphase spread of chromosomes.

18. The method of claim 15, wherein the analyzing comprises comparative genomic hybridization (CGH), fluorescence in situ hybridization (FISH), next generation gene sequencing, and/or giemsa banding.

19. The method of claim 18, wherein the CGH is array CGH.
